(19) United States
(12) Patent Application Publication
(10) Pub. No.: US 2003/0232371 A1
(43) Pub. Date: Dec. 18, 2003

(54) Methods for detecting methylated promoters based on differential DNA methylation

(76) Inventor: Timothy H. Bestor, New York, NY (US)

Correspondence Address: John P. White, Cooper & Dunham LLP, 1185 Avenue of the Americas, New York, NY, 10036, US

(21) Appl. No.: 10/422,566

(22) Filed: Apr. 24, 2003

Related U.S. Application Data
(63) Continuation-in-part of application No. 10/280,375, filed on Oct. 24, 2002.
(60) Provisional application No. 60/346,050, filed on Oct. 24, 2001.

(51) Int. Cl. C12Q 1/68; C12P 19/34

(52) U.S. Cl. 435/6; 435/91.2

(57) ABSTRACT

This invention provides a method for detecting the presence of differential methylation between DNA comprising all or a portion of a promoter from a first source and the corresponding DNA from a second source. Also provided are methods for determining whether a promoter is methylated.

This application is a continuation-in-part of U.S. Ser. No. 10/280,375, filed Oct. 24, 2002, which claims priority of provisional application U.S. Serial No. 60/346,050, filed Oct. 24, 2001, the contents of all of which are incorporated herein by reference.

The invention described herein was made with government support under NIH Grant 1 R01-HGO02425-01. Accordingly, the United States government has certain rights in this invention.

Throughout this application, various references are cited. Disclosure of these references in their entirety is hereby incorporated by reference into this application to more fully describe the state of the art to which this invention pertains.

### BACKGROUND OF THE INVENTION

The mammalian genome contains approximately 3×10⁷ 5-methylcytosine (m⁵C) residues, all or most at 5′-m⁵CpG-3′. About 60% of CpG sites are methylated in the DNA of somatic cells (Bestor et al., 1984; Li et al., 1992). Methylation recruits a variety of transcriptional repressors, including histone deacetylases and other proteins that cause chromosome condensation and silencing (Schübeler et al., 2000; reviewed by Bestor, 1998).

While it has long been known that methylation of a promoter causes profound silencing if the sequence is rich in CpG dinucleotides, only recently have genetic and biochemical experiments begun to identify the biological functions of DNA methylation after many years of controversy and speculation. It was only recently demonstrated that the large majority (>90%) of m⁵C actually lies within intragenomic parasites such as transposons and endogenous retroviruses (which are rich in the CpG dinucleotide and represent more than 45% of the genome; Smit, 1999), and it has been hypothesized that the primary function of cytosine methylation is host-defense against the transcription and dispersal of intragenomic parasites (Bestor, 1990; Bestor and Coxon, 1993; Bestor and Tycko, 1996; Yoder et al., 1997). Allele-specific cytosine methylation has been shown to be required for the monoallelic expression of some imprinted genes. When methylation levels are reduced as a result of homozygous targeted loss-of-function mutations in the Dnmt1 gene, which encodes the major DNA methyltransferase of vertebrates (reviewed by Bestor, 2000), the imprinted genes H19, Igf2, and Igf2r are expressed at equal rates from both parental alleles (Li et al., 1993a; 1993b).

Demethylation of the Xist gene on the X chromosome activates Xist transcription and leads to inactivation of both X chromosomes in female cells and of the sole X in male cells (Panning and Jaenisch, 1996). Other data have shown that demethylation causes fulminating transcription of endogenous retroviral DNA to the point where retroviral transcripts become one of the predominant mRNA species of Dnmt1 mutant embryos (Walsh et al, 1998). However, Dnmt1 mutant embryos do not show ectopic or precocious activation of tissue specific genes, and in fact the promoters of such genes are not normally methylated in non-expressing tissues (Walsh and Bestor, 1999). This suggested that DNA methylation might have primary roles in processes other than reversible gene regulation during development.

There has been much controversy over the biological roles of cytosine methylation. The biological importance of cytosine methylation was long in doubt, in large part because the DNA of familiar laboratory organisms (notably yeast, Drosophila, and C. elegans) lack modified bases. However, genetic studies in mice and humans have shown that abnormalities of genomic methylation patterns have severe phenotypic consequences. Disruption of the Dnmt1 gene (Bestor et al., 1988) showed that demethylation of the genome caused apoptotic cell death in all differentiating cell types, fulminating expression of normally silenced retroposons, loss of imprinted expression at a number of imprinted loci, ectopic X inactivation, and marked chromosome instability manifested as a high rate of deletions and rearrangements (reviewed by Bestor, 2000).

Human genetic disorders were recently shown to be caused by mutations in a DNA methyltransferase gene (Xu et al., 1999) and in a gene that encodes a protein that binds to methylated DNA (Amir et al., 1999). The first of these, ICF syndrome, is characterized by immunodeficiency, centromere instability, and facial anomalies. The cytogenetic abnormalitIes are extreme; chromosomes 1, 9, and 16 gain and lose short arms such that a single chromosome can have as many as 12 short arms. The resulting pinwheel chromosomes are highly diagnostic. The breakage and rejoining occurs at tracts of classical satellite DNA, which is normally heavily methylated but is completely unmethylated in DNA of ICF patients. It has been shown that ICF syndrome is due to inactivating point mutations in the DNMT3B gene on chromosome 20 (Xu et al., 1999).

The second syndrome, Rett syndrome, is a common neurodevelopmental syndrome in which normal early development is followed by a regression in all neural functions leading to complete apraxia and death by aspiration pneumonia or heart failure. The syndrome is due to mutations in MeCP2, which encodes a transcriptional repressor that binds specifically to methylated DNA (Amir et al., 1999).

Methylation abnormalities have also been seen in patients suffering from ATRX (alpha thalassemia and mental retardation on the X) syndrome (Gibbons et al., 2000). The genetic findings in both mice and humans confirm that cytosine methylation has multiple essential roles. There is, however, much remaining uncertainty and continuing controversy as to the nature of those roles.

Another aspect of genomic methylation patterns is the frequent finding of ectopic de novo methylation of CpG islands associated with tumor suppressor genes in human tumors and tumor cells lines (reviewed by Warnecke and Bestor, 2000). First observed at RB1, ectopic promoter methylation has come to be regarded as a common mechanism by which tumor suppressor genes are inactivated in cancer. However, there is little direct evidence that the observed methylation is responsible for the silencing, and most studies have used DNA from cultured tumor cell lines in which genomic methylation patterns are very unstable. Nonetheless, the high frequency with which promoter methylation is observed at tumor suppressor loci indicates the possibility that this feature can be used to identify candidate tumor suppressor genes that might not be identified through other means.

Given that inherited and somatic changes in methylation patterns are involved in human disease, it is unfortunate that so little should be known of the basic organization of genomic methylation patterns. The methylation landscape of the human genome, as well as the role of methylation pattern dynamics in normal development, carcinogenesis, and human genetic disorders remains an important area for exploration. Unfortunately, there remains a need for experimental methods suitable for investigating methylation's role in the genome.

### SUMMARY OF THE INVENTION

This invention provides a first method for detecting the presence of differential methylation between DNA comprising all or a portion of a promoter from a first source and the corresponding DNA from a second source, which method comprises the steps of

(a) (i) contacting an agent that degrades methylated DNA with a sample of the DNA from the first source, under suitable conditions, so as to degrade methylated DNA in the first sample, and (ii) contacting an agent that degrades unmethylated DNA with a sample of the DNA from the second source, under suitable conditions, so as to degrade unmethylated DNA in the second sample;

(b) contacting the resulting samples with each other under conditions permitting reannealing between the DNA strands therein, so as to permit the formation of a hybrid DNA duplex comprising a DNA strand from the first source and a DNA strand from the second source, should both such strands be present; and

(c) detecting the formation of any such hybrid DNA duplex, such formation indicating the presence of differential methylation between the DNA from the first source and the corresponding DNA from the second source.

This invention also provides a second method for determining whether a promoter is methylated, which method comprises the steps of

(a) (i) contacting an agent that degrades unmethylated DNA with a first sample of DNA comprising all or a portion of the promoter, under suitable conditions, so as to degrade unmethylated DNA in the sample, and (ii) contacting an agent that degrades methylated DNA with a second sample of DNA corresponding to the first, under suitable conditions, so as to degrade methylated DNA in the sample, wherein the DNA in the second sample is known to be unmethylated;

(b) contacting the resulting samples with each other under conditions permitting reannealing between the DNA strands therein, so as to permit the formation of a hybrid DNA duplex comprising a DNA strand from the first sample and a DNA strand from the second sample, should both such strands be present; and

(c) detecting the formation of any such hybrid DNA duplex, such formation indicating the presence of differential methylation between the DNA from the first sample and the corresponding DNA from the second, and hence indicating that the promoter is methylated.

This invention also provides a third method for determining whether a promoter is methylated, which method comprises the steps of

(a) contacting a first sample of DNA comprising all or a portion of the promoter with an agent that degrades unmethylated DNA, under suitable conditions, so as to degrade unmethylated DNA in the sample;

(b) contacting the resulting first sample with a second sample of corresponding DNA, known to be unmethylated, under conditions permitting reannealing between the DNA strands therein, so as to permit the formation of a hybrid DNA duplex comprising a DNA strand from the first sample and a DNA strand from the second sample, should both such strands be present; and

(c) detecting the formation of any such hybrid DNA duplex, such formation indicating the presence of differential methylation between the DNA from the first sample and the corresponding DNA from the second, and hence indicating that the promoter is methylated.

### BRIEF DESCRIPTION OF THE FIGURES

FIG. 1

Organization of transposons, exons, and HpaII (CCGG) sites within the human HPRT gene. Organization of HPRT is typical of human genes (Yoder et al., 1997). CCGG sites located in known transposons and in cellular sequences are shown in contrasting shades; note the concentration of cellular CCGG sites in the CpG island at the 5′ end of the gene. Nearly all of the CCGG sites within the body of the gene are in transposons. As shown by the scale at right the gene is methylated at these sites and unmethylated at the CpG island, as is true of the large majority of cellular genes. The CpG island undergoes dense de novo methylation when located on the inactive X chromosome, but is completely unmethylated on the active X (Litt et al., 1996). CCGG sites are shown here as they are most often used to evaluate methylation patterns by Southern blot analysis.

FIGS. 2A-2C

Removal of methylated sequences by McrBC digestion and of unmethylated sequences by RE digestion. (2A) Unmethylated S. pombe DNA was resistant to McrBC digestion (lane 3); after methylation of all CpG sites by treatment with M.SssI it became very sensitive (lane 4). Unmethylated S. pombe DNA is very sensitive to RE treatment (lane 5; discrete bands were derived from very G+C-poor mitochondrial DNA). (2B) McrBC-resistant fragments in human Jurkat test DNA (lane 2) were sensitive to RE treatment, indicating that they were in fact unmethylated in the starting DNA and did contain CpG sites. (2C) Methylation of human DNA at all CpG sites with M. SssI shows that the McrBC-resistant fraction>500 bp in lane 5 is unmethylated, as shown by the acquisition of McrBC sensitivity after M. SssI treatment (lane 3). Gap below 500 bp in all panels is artifact of bromphenol blue.

FIG. 3

Removal of endogenous methylated sequences from McrBC libraries. LINE-1 (L1) elements (left) and satellite 3 DNA are normally heavily methylated. The figure shows that these sequences are largely removed from human DNA by digestion with McrBC. The size range is set between the lower limit for CpG islands (˜500 bp) and the upper limit for clonability in plasmid vectors.

### DETAILED DESCRIPTION OF THE INVENTION

Definitions

As used in this application, except as otherwise expressly provided herein, each of the following terms shall have the meaning set forth below.

As used herein, a first DNA which “corresponds” to a second DNA preferably has the same nucleotide sequence as the second DNA. A first sample of DNA which “corresponds” to a second sample of DNA preferably contains DNA having the same nucleotide sequence as the DNA in the second sample.

“Normal cell corresponding to a tumor cell” shall mean a non-diseased cell of the same type as that from which the tumor cell originated.

As used herein, “promoter” shall mean a sequence of nucleotides on DNA that is required for the initiation of transcription by RNA polymerase. Promoters include, without limitation, promoters of gene transcription. In one embodiment, the promoter is not normally methylated due to imprinting.

“Source of DNA” includes, but is not limited to, a normal tissue, a diseased tissue, a cell, a virus, and populations thereof, a biological fluid sample, a cultured cell or population thereof, a tissue or cell biopsy, a pathological sample, a forensic sample, a chromosome, chromatin, genomic DNA, a DNA library and an isolated gene.

As used herein, “subject” means any animal or artificially modified animal. Animals include, but are not limited to, mice, rats, dogs, guinea pigs, ferrets, rabbits, and primates. In the preferred embodiment, the subject is a human.

Embodiments of the Invention

This invention provides a first method for detecting the presence of differential methylation between DNA comprising all or a portion of a promoter from a first source and the corresponding DNA from a second source, which method comprises the steps of

(a) (i) contacting an agent that degrades methylated DNA with a sample of the DNA from the first source, under suitable conditions, so as to degrade methylated DNA in the first sample, and (ii) contacting an agent that degrades unmethylated DNA with a sample of the DNA from the second source, under suitable conditions, so as to degrade unmethylated DNA in the second sample;

(b) contacting the resulting samples with each other under conditions permitting reannealing between the DNA strands therein, so as to permit the formation of a hybrid DNA duplex comprising a DNA strand from the first source and a DNA strand from the second source, should both such strands be present; and

(c) detecting the formation of any such hybrid DNA duplex, such formation indicating the presence of differential methylation between the DNA from the first source and the corresponding DNA from the second source.

In one embodiment, the first method further comprises the step of modifying the DNA of parts (i) and (ii) resulting from step (a) with a first and second moiety, respectively, so as to prevent, in step (b), the formation of a DNA duplex consisting of DNA strands from the first source or of a DNA duplex consisting of DNA strands from the second source. In one example, the modification of at least one sample resulting from step (a) comprises modifying the DNA in at least one sample with a moiety which facilitates the isolation of hybrid DNA duplexes formed in step (b). Such moieties are well known in the art and include, for example, biotin.

In another embodiment, the first method further comprises the step of determining the nucleic acid sequence of a hybrid DNA duplex whose presence is detected in step (c). In one example, this step further comprises the step of identifying the methylated nucleotide residues of one or both strands of the hybrid DNA duplex whose sequence is determined.

In the first method, the first and second sources of DNA can be any suitable sources such as, for example, (i) a cell from a first tissue of a subject and a cell from a second tissue of that subject, respectively; (ii) a cell from a normal tissue and a cell from that tissue in a diseased state, respectively; (iii) chromosomes of a chromosome pair; and (iv) all or a portion of a promoter. In a preferred embodiment, the promoter is a promoter for a tumor suppressor gene. In another preferred embodiment, the promoter is a promoter for an oncogene.

In another embodiment of the first method, the agent that degrades methylated DNA is McrBC. In another embodiment, the agent that degrades unmethylated DNA comprises a methylation-sensitive restriction endonuclease. In one embodiment, the methylation-sensitive restriction endonuclease is selected from the group consisting of HpaII, HhaI, MaeII, BstUI and AciI. In a further embodiment, the agent that degrades unmethylated DNA comprises a plurality of methylation-sensitive restriction endonucleases. Preferably, the plurality of methylation-sensitive restriction endonucleases is selected from the group consisting of HpaII, HhaI, MaeII, BstUI and AciI.

In the preferred embodiment, the DNA from the first and second sources is human DNA.

This invention also provides a second method for determining whether a promoter is methylated, which method comprises the steps of

(a) (i) contacting an agent that degrades unmethylated DNA with a first sample of DNA comprising all or a portion of the promoter, under suitable conditions, so as to degrade unmethylated DNA in the sample, and (ii) contacting an agent that degrades methylated DNA with a second sample of DNA corresponding to the first, under suitable conditions, so as to degrade methylated DNA in the sample, wherein the DNA in the second sample is known to be unmethylated;

(b) contacting the resulting samples with each other under conditions permitting reannealing between the DNA strands therein, so as to permit the formation of a hybrid DNA duplex comprising a DNA strand from the first sample and a DNA strand from the second sample, should both such strands be present; and

(c) detecting the formation of any such hybrid DNA duplex, such formation indicating the presence of differential methylation between the DNA from the first sample and the corresponding DNA from the second, and hence indicating that the promoter is methylated.

In one embodiment, the second method further comprises the step of modifying the DNA of parts (i) and (ii) resulting from step (a) with a first and second moiety, respectively, so as to prevent, in step (b), the formation of a DNA duplex consisting of DNA strands from the first sample or of a DNA duplex consisting of DNA strands from the second sample. In one example, the modification of at least one sample resulting from step (a) comprises modifying the DNA in at least one sample with a moiety which facilitates the isolation of hybrid DNA duplexes formed in step (b). Such moieties are well known in the art and include, for example, biotin.

In another embodiment, the second method further comprises the step of determining the nucleic acid sequence of a hybrid DNA duplex whose presence is detected in step (c). In one example, this step further comprises the step of identifying the methylated nucleotide residues of one or both strands of the hybrid DNA duplex whose sequence is determined.

In another embodiment of the second method, the agent that degrades methylated DNA is McrBC. In another embodiment, the agent that degrades unmethylated DNA comprises a methylation-sensitive restriction endonuclease. In one embodiment, the methylation-sensitive restriction endonuclease is selected from the group consisting of HpaII, HhaI, MaeII, BstUI and AciI. In a further embodiment, the agent that degrades unmethylated DNA comprises a plurality of methylation-sensitive restriction endonucleases. Preferably; the plurality of methylation-sensitive restriction endonucleases is selected from the group consisting of HpaII, HhaI, MaeII, BstUI and AciI.

In the preferred embodiment, the DNA from the first and second samples is human DNA. In another preferred embodiment, the first sample is from a human being known to be afflicted with a disorder. In a further embodiment, the disorder is cancer.

In another embodiment of the second method, the promoter is a promoter for a tumor suppressor gene. In another embodiment, the promoter is a promoter for an oncogene.

In another embodiment of the second method, the first and second samples consist of all or a portion of a promoter.

This invention also provides a third method for determining whether a promoter is methylated, which method comprises the steps of

(a) contacting a first sample of DNA comprising all or a portion of the promoter with an agent that degrades unmethylated DNA, under suitable conditions, so as to degrade unmethylated DNA in the sample;

(b) contacting the resulting first sample with a second sample of corresponding DNA, known to be unmethylated, under conditions permitting reannealing between the DNA strands therein, so as to permit the formation of a hybrid DNA duplex comprising a DNA strand from the first sample and a DNA strand from the second sample, should both such strands be present; and

(c) detecting the formation of any such hybrid DNA duplex, such formation indicating the presence of differential methylation between the DNA from the first sample and the corresponding DNA from the second, and hence indicating that the promoter is methylated.

In one embodiment, the third method further comprises the step of modifying the DNA of the first and second samples with a first and second moiety, respectively, so as to prevent, in step (b), the formation of a DNA duplex consisting of DNA strands from the first sample or of a DNA duplex consisting of DNA strands from the second sample. In one embodiment, the modification comprises modifying the DNA in at least one sample with a moiety which facilitates the isolation of hybrid DNA duplexes formed in step (b). Such moieties are well known in the art and include, for example, biotin.

In another embodiment, the third method further comprises the step of determining the nucleic acid sequence of a hybrid DNA duplex whose presence is detected in step (c). In one example, this step further comprises the step of identifying the methylated nucleotide residues of a strand of the hybrid DNA duplex whose sequence is determined.

In another embodiment of the third method, the agent that degrades unmethylated DNA comprises a methylation-sensitive restriction endonuclease. In one embodiment, the methylation-sensitive restriction endonuclease is selected from the group consisting of HpaII, HhaI, MaeII, BstUI and AciI. In a further embodiment, the agent that degrades unmethylated DNA comprises a plurality of methylation-sensitive restriction endonucleases. Preferably, the plurality of methylation-sensitive restriction endonucleases is selected from the group consisting of HpaII, HhaI, MaeII, BstUI and AciI.

In the preferred embodiment, the DNA from the first and second samples is human DNA. In another preferred embodiment, the first sample is from a human being known to be afflicted with a disorder. In a further embodiment, the disorder is cancer.

In another embodiment of the third method, the promoter is a promoter for a tumor suppressor gene. In another embodiment, the promoter is a promoter for an oncogene.

In another embodiment of the third method, the first and second samples consist of all or a portion of a promoter.

This invention will be better understood from the Experimental Details that follow. However, one skilled in the art will readily appreciate that the specific methods and results discussed are merely illustrative of the invention as described more fully in the claims which follow thereafter.

Experimental Details

Background

Host Defense Hypothesis

Applicants were the first to purify, characterize, and clone a eukaryotic DNA methyltransferase (Dnmt1; Bestor et al., 1988). Applicants also disrupted the Dnmt1 gene (in collaboration with R. Jaenisch) and demonstrated that cytosine methylation is essential for mammalian development (Li et al., 1992). Several of the biological functions of cytosine methylation have been deduced from studies of Dnmt1 mutant mice. The Dnmt1 gene was the first gene shown to have sex-specific promoters and first exons (Mertineit et al., 1998), and deletion of the female-specific promoter and first exon was the first pure maternal-effect mutation to be observed in a mammal (Howell et al., 2001). Applicants also found the first human genetic disorder to be caused by mutations in a DNA methyltransferase gene (Xu et al., 1999), and were the first to solve the crystal structure of a eukaryotic DNA methyltransferase homologue, human DNMT2 (Dong et al., 2001), whose function is unknown and is currently under study.

Applicants also put forward the idea that the primary function of cytosine methylation is likely to be host defense against transposons (Bestor, 1990; Yoder et al., 1997; Bestor, 2000). The host defense hypothesis has come to be supported by a large body of evidence and has received increasingly positive regard after a somewhat emotional reception by colleagues devoted to the developmental hypothesis. However, until more is known of the large-scale patterning of cytosine methylation in the genome, there will be continuing controversy as to the biological functions of methylation patterns.

The Shape of Genomic Methylation Patterns

Cytosine methylation is erased by cloning in microorganisms or by PCR amplification and information on methylation patterns is therefore absent from the human genome sequences produced by both the public and private sequencing efforts.

Current methods for the analysis of cytosine methylation are ineffective. These methods involve Southern blot analysis after cleavage with methylation-sensitive restriction endonucleases or PCR across the restriction sites of such enzymes, or the sequencing of genomic DNA after deamination by sodium bisulfite treatment, which converts all cytosines to uracils but does not convert m⁵C so that all remaining cytosines must have been derived from m⁵C.

These traditional methods have inherent limitations appropriate to their pre-genomics beginnings; they are very limited in scope and can be used to test only small regions, they require that the sequences be known in advance and cannot be used to extract sequences that are heavily methylated or largely unmethylated, and the Southern blot method (which is most widely used) can examine only a few sites with narrow spacing requirements. It is the CpG density and methylation status of regions of hundreds of base pairs, rather than single CpG sites, that appear to control promoter activity (Kass et al., 1997). Examination of single sites, which are usually chosen on the basis of convenience, can therefore be quite misleading. In addition to these technical issues, it is common to work with DNA from established lines of cultured cells rather than tissue DNA. Genomic methylation patterns are highly unstable in cultured cells, and in cell lines the promoters of tissue-specific genes are frequently methylated at positions that are not methylated in non-expressing tissues. The muscle-specific α-actin gene, for example, is methylated in most mouse and human cell lines but is not methylated in mouse brain, liver, or spleen, tissues that do not express α-actin (Walsh and Bestor, 1999).

Although the extant data are fragmentary and often contradictory, a few themes do emerge repeatedly. First, promoter regions that are heavily methylated in tissues are normally silent (examples are imprinted genes and those on the inactive X chromosome in females, and promoters that have undergone de novo methylation in cultured cells or tumors). Second, CpG islands (regions of high G+C content and CpG density which span or overlap the 5′ ends of most genes) are unmethylated in the germ line and in all somatic tissues, except when associated with imprinted genes or those subject to X inactivation. Third, gene silencing usually involves methylation of all or nearly all CpG sites in CpG islands that are 500-2,000 base pairs in length; methylation of non-CpG island sequences does not usually prevent transcription (Kass et al., 1997), and the binding of transcription factors can actually cause demethylation of local CpG sites (Lin et al., 2000). Fourth, the large majority of genomic m⁵C is within transposons, which are abundant (45% of the mammalian genome; Smit, 1999) and relatively rich in CpG dinucleotides. More than 90% of genomic m⁵C lies with retroposons (Yoder et al., 1997), and other repeated sequences such as pericentric satellite DNA account for much of the remainder. However, it must be kept in mind that the regulatory regions of cellular genes represent much less than 1% of the total genome, and this small contribution will not be detectable against the large background of heavily methylated transposons and other repeated sequences.

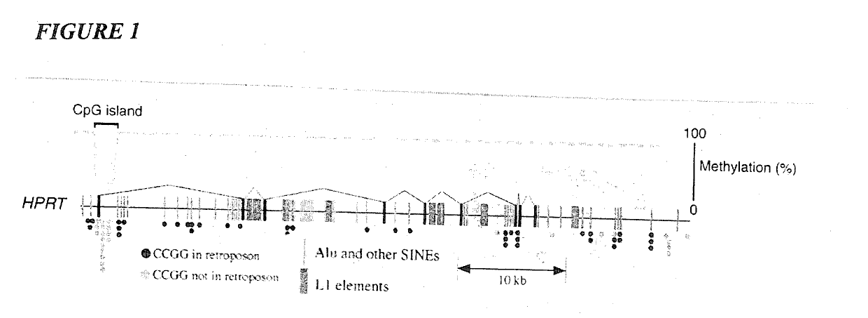

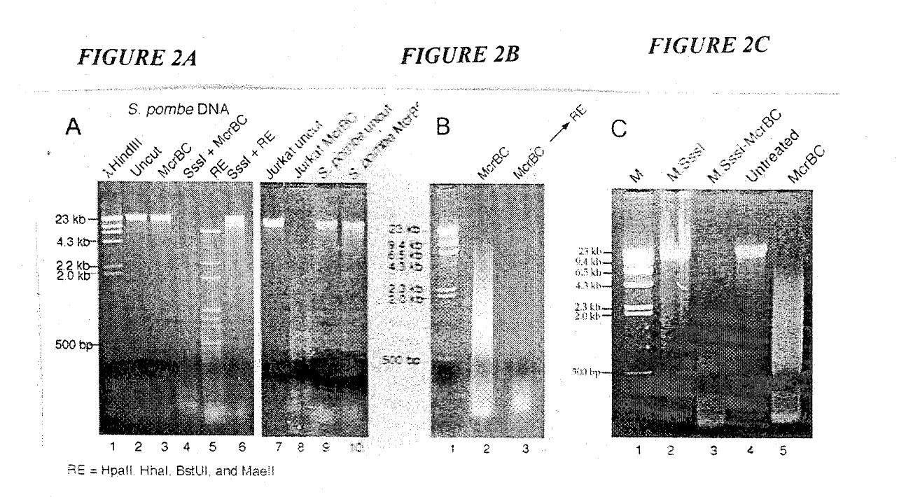

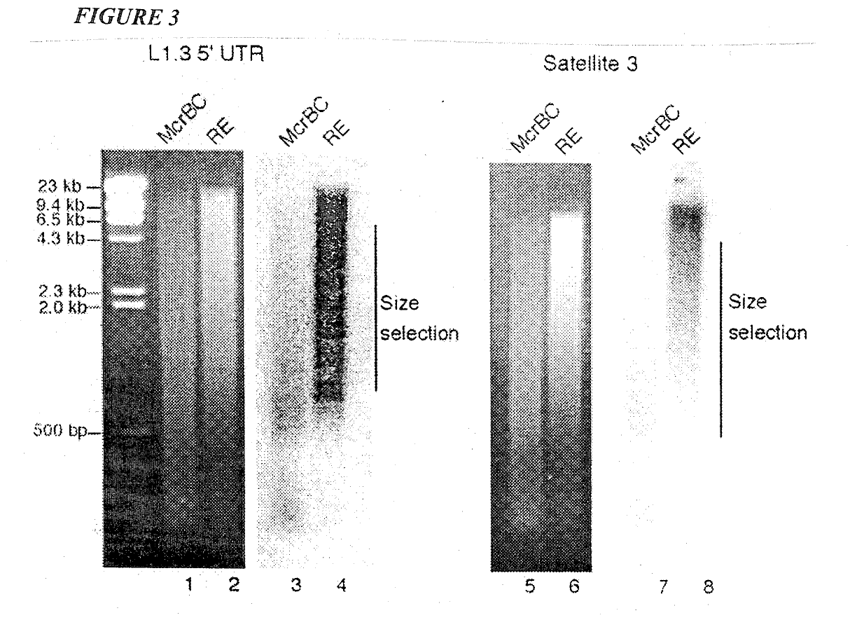

Most genes have unmethylated promoters in both expressing and non-expressing tissues, although the transcribed regions tend to be methylated. That is because introns are rich in transposons, which are largely methylated. This is illustrated by examination of the HPRT gene in FIG. 1. The genome browser (http://genome.ucsc.edu/goldenPath/septTracks.html; please note that all references to “genome browser” refer to this software) annotates transposon distributions, and all long genes can be seen to contain multiple transposons. Some, such as VHL, are more than 50% transposon.

While the above suggests that the genome is characterized by unmethylated single copy cellular sequences embedded in a background of methylated transposons, the situation is actually more complex. CpG sites in exons can be heavily methylated if they lie close to transposons in flanking introns. Such CpG sites are especially vulnerable to C→T transition mutations driven by deamination of m⁵C (Magewu and Jones, 1994). CpG islands can be heavily methylated in normal cells, as in the case of imprinted genes and those subject to X inactivation, and much demethylation (Feinberg and Vogelstein, 1983) and de novo methylation is seen in DNA of cancer cells (reviewed by Warnecke and Bestor, 2000). Stochastic and ectopic de novo methylation has been attributed a role in human disorders in which there appear to be both genetic and epigenetic contributions to phenotype (Petronis et al., 2000). However, once again the lack of knowledge of the large-scale patterning of m⁵C in the genome, and the lack of a known method for the extraction of differentially methylated sequences, has engendered controversy and slowed progress.

Methods and Results

Experiment I

Applicants have developed methods for the selective cloning of the heavily methylated compartment and the unmethylated compartment of the genome. The methylated compartment is resistant to methylation-sensitive restriction endonucleases. Applicants use a mixture of 5 such enzymes (HpaII, C*CGG; MaeII, A*CGT; BstUI, *CG*CG, HhaI, G*CGC, and AciI, CC*GC and G*CGG; asterisk identifies site of methylation that prevents cleavage). The unmethylated compartment is resistant to McrBC, an E. coli enzyme complex that binds to sequences of the form Rm⁵C—(N)₄₀₋₅₀₀-Rm⁵C and degrades all internal sequences to small fragments (Stewart and Raleigh, 1998). Little degradation is seen when the two half-sites are more than 500 base pairs apart. The unmethylated sequences in most CpG islands are greater than 500 base pairs (Cross et al., 2000). The genome browser flags CpG islands of <400 base pairs as questionable based on length alone.

To confirm the reported behavior of the enzymes, applicants first treated the unmethylated DNA of Schizosaccharomyces pombe with McrBC (New England Biolabs) or with the mixture of methylation-sensitive restriction endonucleases (referred to as “RE treatment”). As shown in FIGS. 2A-2C, the unmethylated DNA was completely resistant to McrBC, but was degraded to small fragments by RE treatment (lanes 3 and 5; bands in lane 5 are from mitochondrial DNA, which is A+T-rich and poor in CpG dinucleotides). When S. pombe DNA was methylated at all CpG sites by in vitro treatment with the DNA methyltransferase M.SssI (New England Biolabs) and S-AdoMet, it was rendered completely resistant to RE (lane 6) treatment but became very sensitive to McrBC (lane 4). The DNA of cultured Jurkat cells (a human T cell leukemia cell line) was sensitive to McrBC, but markedly less so than artificially methylated S. pombe DNA, which has no unmethylated compartment (lanes 4 and 8). These test data confirm that McrBC and RE treatment have the expected effects on methylated and unmethylated sequences.

Even though McrBC has relaxed sequence and spacing requirements, it was of concern that the McrBC-resistant fraction shown above may have been derived from methylated DNA that has a very low CpG density and therefore lacks half sites in the configuration required for McrBC digestion. If this were so, the McrBC-resistant fraction would also be RE resistant as a result of methylation or sparse CpG sites. As shown in FIG. 2B, the McrBC-resistant fraction is very sensitive to RE treatment, and FIG. 2C shows that methylation of CpG sites converts the McrBC resistant fraction to McrBC-sensitive. These data confirm that the McrBC library is composed largely of unmethylated CpG-containing sequence tracts.

Applicants next confirmed that sequence compartments in the human genome that are known to be heavily methylated can be eliminated by McrBC digestion but resist RE treatment. Applicants chose the promoter region of a LINE-1 element, L1.3, that has been shown to belong to a family of actively transposing L1 elements (reviewed by Kazazian and Moran, 1998). These have been found to be heavily methylated in all cell types examined. Also tested was classical satellite 3 DNA from chromosome 9, which is densely methylated in all normal cells but is unmethylated in patients with ICF syndrome and in certain tumor types (Xu et al., 1999). As shown in FIG. 3, the specific methylated sequences could be almost completely removed by McrBC treatment. Applicants have prepared plasmid libraries of human genomic DNA restricted by McrBC or by RE treatment. A size selection is performed as indicated in FIG. 3 to reduce the already low background, and the DNA is cloned into the EcoRV site of pZErO-1 zero background cloning vector (Invitrogen) after blunting insert ends with T4 DNA polymerase. These McrBC libraries will be depleted in heavily methylated sequences, while the RE libraries will be enriched in such sequences.

These data show that the instant methods allow the selective cloning of both the unmethylated and heavily methylated compartments of the genome. Sequence analysis of McrBC and RE libraries permits the first objective large-scale view of the methylation landscape of the human genome. These data also facilitate the identification of CpG islands by objective criteria. The present computational methods must use arbitrary thresholds for CpG density and G+C content and tend to overestimate CpG island number by a factor or 2. For example, distal 21q contains 110 genes, but 234 predicted CpG islands. It seems unlikely that gene number was underestimated by a factor of 2. Subtractive hybridization of the McrBC and RE libraries permits selective extraction of sequences that are differentially methylated between normal and cancer cells, between tissues of normal individuals and those with genetic disorders such as Rett and ICF syndromes, and between alleles in the case of imprinted genes. All these data can be analyzed on-line by new computational methods and added as annotation to the human genome browser in a fully automated and almost real-time basis.

Experiment II

It is commonly held that the function of genomic methylation patterns is the control of gene expression during development and that promoters are unmethylated only in tissues that express the associated gene (Venter et al., 2001). It is believed that most promoters in normal tissues are methylated. However, in the art, no large-scale and unbiased analysis of promoter methylation has been conducted.

Applicant applied methods for fractionation of the genome into methylated and unmethylated compartments and mapped 3,144 unmethylated and 1,400 methylated domains onto the human genome. Applicant found that 400 promoters were within unmethylated domains and only one promoter (that of the SCP3 gene, which may have been an artifact) was within a methylated domain.

These data indicate that promoter methylation is much less frequent than is commonly believed and that the presence of a methylated promoter, especially when that promoter has a content of guanosine plus cytosine of >50%, is likely to reflect a pathological state. The finding that a promoter of a gene is methylated in a diseased tissue is an indication that a loss of expression of that gene contributes to the specific disease. This is based on the novel finding that, contrary to the prevailing perception in the field of molecular biology, the large majority of human promoters are not methylated regardless of whether they are expressed in the tissue from which the DNA was purified.

Discussion

In the mammalian genome, DNA methylation occurs predominantly at cytosine residues found in the context of CpG dinucleotides. In contrast to genetic alterations, cytosine methylation is an epigenetic modification, which is potentially reversible and does not alter DNA sequence. As the most well characterized mechanism of epigenetic-regulation, DNA methylation has been implicated in a number of biological processes, including genomic imprinting, X-inactivation, and silencing of parasitic DNA. Abnormal cytosine methylation is thought to contribute to disease states, as aberrant genomic methylation patterns have been observed in cancer and genetic disorders, such as ICF Syndrome and Rett Syndrome, as well as schizophrenia. Demethylation also destabilizes the genome and can contribute to the development of cancer. Given the deleterious effects of aberrant DNA methylation, it is surprising how little is known about normal methylation patterns in the mammalian genome. This is due in part to the lack of efficient methods for the identification of regions of the genome that differ in methylation status between cell types. Such a method would be very powerful in the identification of tumor suppressors; once identified, such new tumor suppressors become targets of rational drug design.

Until recently, the analysis of altered methylation patterns has been limited to a small number of predetermined genes. Traditional molecular biology techniques, such as Southern blotting and polymerase chain reaction (PCR), are not capable of analyzing global methylation patterns and they cannot be used to isolate sequences on the basis of abnormal methylation status. The more recent use of Restriction Landmark Genome Scanning (RLGS) and Methylation-Sensitive Representational Difference Analysis (MS-RDA) have met with limited success. RLGS is a cumbersome, labor-intensive method in which methylation changes are visualized as a dense cluster of “spots” on 2-dimensional gels. This poor resolution presents major difficulties in the identification and isolation of genomic loci, making it unsuitable for high-throughput. MS-RDA is a PCR-based technique that is biased toward short DNA fragments and against GC-rich sequences. Novel array-based methods have also been developed, but these rely heavily on hybridization kinetics. All existing methods are vulnerable to the presence of normal cells in the diseased tissue. With the increasing emphasis on the potential role of methylation in human diseases, there is an immediate need for an effective method for identifying genome-wide changes in DNA methylation in human tissue samples.

To meet this need, applicants have developed a novel method for identifying alterations in DNA methylation. This procedure, which applicants refer to as Methylation Subtraction Analysis (MSA), relies on the enzymatic fractionation of the human genome into its methylated and unmethylated compartments. This fractionation method coupled with standard molecular biology techniques facilitates the identification and isolation of genomic sequences that are unmethylated in normal tissue but have become hypermethylated in disease tissue.

MSA offers several key advantages over other techniques for identifying global changes in DNA methylation. Most importantly, genomic DNA used in this procedure can be obtained directly from normal and disease tissues rather than cultured cell lines. This point is underscored by the recent observation that more than 57% of sequences found to be methylated in cultured tumor cells were not methylated in the corresponding primary tumors. In some tumors, the error rate is 97% (Smiraglia et al., 2001). Another advantage of MSA is that it is insensitive to contamination of tumor samples by normal cells. One of the difficulties in analyzing tumor samples, for instance, is that the tumors themselves are often a heterogeneous mix of wild-type and cancerous cells. MSA has been designed so that methylated sequences from disease cells will be enzymatically removed from unmethylated genomic libraries derived from normal tissue while unmethylated sequences will be enzymatically removed from methylated libraries derived from disease tissue. This allows for accurate identification of genomic loci that display differential methylation between the normal and disease tissues. Finally, the robust and streamlined nature of the MSA procedure makes it ideal for high-throughput analyses of genome-wide methylation differences. Since the final readout is actual DNA sequence, MSA avoids the tedious cloning of individual candidate loci, which is a major obstacle to high-throughput analysis.

From a commercialization standpoint, the MSA procedure has several research and clinical applications. Several tumor-suppressor genes have been identified based on the observation that they are aberrantly methylated in cancerous cells. This number, however, is an underestimation, primarily due to the limitations of existing methods for analyzing genome-wide methylation changes. To this end, MSA is well suited for the identification of new tumor-suppressor genes as well genes that may contribute to other human disorders. Newly identified genes may serve as targets for future therapies that focus on targeted demethylation. By a simple modification of the fractionation procedure, MSA can also detect the loss of methylation. This can be used to identify new oncogenes that are normally silenced by methylation but have become activated during the oncogenic process. The proteins encoded by these genes may be potential drug targets that drive the development of new treatments.

While methylation status of a genomic locus does not always signify its involvement in a particular disease, the methylation patterns themselves undoubtedly have diagnostic and prognostic value in the treatment of disease. For example, certain tumor types may have different hypermethylation profiles during the course of tumor progression. These tumor-specific profiles can facilitate early cancer diagnosis as well as cancer prognosis. MSA is well suited for the large-scale extraction of sequences subject to aberrant methylation in human cancer. Methylation analysis is an entirely new route to the identification of tumor suppressors.

References

Amir R E, Van den Veyver I B, Wan M, Tran C Q, Francke U, Zoghbi H Y (1999) Rett syndrome is caused by mutations in X-linked MECP2, encoding methyl-CpG-binding protein 2. Nat. Genet. 23, 185-188.

Antequera F, Bird A (1993) Number of CpG islands and genes in human and mouse. Proc. Natl. Acad. Sci. USA 90, 11995-11999.

Bestor T H (1990) DNA methylation: Evolution of a bacterial immune function into a regulator of gene expression and genome structure in higher eukaryotes. Phil. Trans. Royal Soc. Lond. B 326, 179-187.

Bestor T H (1998) Gene silencing. Methylation meets acetylation. Nature 393, 311-312.

Bestor T H (2000) The DNA methyltransferases of mammals. Hum. Mol. Gen. 9, 2395-2402.

Bestor T H (2000) Sex brings transposons and genomes into conflict. Genetica 207, 289-295.

Bestor T H (2000) Gene silencing as a threat to the success of gene therapy. J. Clin. Invest. 105, 409-411.

Bestor T H, Coxon A (1993) The pros and cons of DNA methylation. Curr. Biol. 3, 384-386.

Bestor T H, Hellewell S B, Ingram V M (1984) Differentiation of Two Mouse Cell Lines Is Associated with Hypomethylation of Their Genomes. Mol. Cell. Biol. 4, 1800-1806.

Bestor T, Laudano A, Mattaliano R, Ingram V (1988) Cloning and sequencing of a cDNA encoding DNA methyltransferase of mouse cells. The carboxyl-terminal domain of the mammalian enzyme is related to bacterial restriction methyltransferases. J. Mol. Biol. 203, 971-983.

Bestor T H, Tycko B (1996) Creation of genomic methylation patterns. Nature Genetics 12, 363-367.

Caspary T, Cleary M A, Baker C C, Guan X J, Tilghman S M (1998) Multiple Mechanisms Regulate Imprinting of the Mouse Distal Chromosome 7 Gene Cluster. Mol. Cell. Biol. 18, 3466-74.

Costello J F, Fruhwald M C, Smiraglia D J, Rush L J, Robertson G P, Gao X, Wright F A, Feramisco J D, Peltomaki P, Lang J C, Schuller D E, Yu L, Bloomfield C D, Caligiuri M A, Yates A, Nishikawa R, Su Huang H J, Petrelli N J, Zhang X, O'Dorisio M S, Held W A, Cavenee W K, Plass C (2000) Aberrant CpG-island methylation has non-random and tumour-type-specific patterns. Nat. Genet. 25, 132-138.

Cross S H, Clark V H, Simmen M W, Bickmore W A, Maroon H, Langoford C F, Carter N P, Bird A P (2000) CpG island libraries from human Chromosomes 18 and 22: landmarks for novel genes. Mamm. Genome 11, 373-383.

Dong A, Yoder J A, Zhang X, Zhou L, Bestor T H, and Cheng X (2001) Structure of Human DNMT2, and enigmatic DNA methyltransferase homologue that displays denaturant-resistant binding to DNA. Nucl. Acids Res. 29, 439-448.

Feinberg A P and Vogelstein B (1983) Hypomethylation distinguishes genes of some human cancers from their normal counterparts. Nature 301, 89-92.

Gibbons R J, McDowell T I, Raman S., O'Rourke D M, Garrick D., Ayyub H. Higgs D R (2000) Mutations in ATRX, encoding a SWI/SNF-like protein, cause diverse changes in the pattern of DNA methylation. Nat. Genet. 24, 368-371.

Howell C Y, Bestor T H, Deng F, Latham K E, Mertineit C, Trasler J M, Chaillet J R (2001) Genomic Imprinting Disrupted by a Maternal Effect Mutation in the Dnmt1 Gene. Cell 104, 829-838.

Jaakkola T, Diekhans M, Haussler D (2000) A Discriminative Framework for Detecting Remote Protein Homologies. J. Comput. Biol. 7, 95-114.

Kass S U, Landsberger N, Wolffe A P (1997) DNA methylation directs a time-dependent repression of transcription initiation. Curr. Biol: 7, 157-165.

Kazazian H H Jr, Moran J V (1998) The impact of L1 retrotransposons on the human genome. Nat. Genet. 19, 19-24.

Kuromitsu J, Yamashita H, Kataoka H, Takahara T, Muramatsu M, Sekine T, Okamoto N, Furuichi Y, Hayashizaki Y (1997) A Unique Downregulation of h2-Calponin Gene Expression in Down Syndrome: a Possible Attenuation Mechanism for Fetal Survival by Methylation at the CpG Island in the Trisomic Chromosome 21. Mol. Cell. Biol. 17, 707-712.

Li E, Beard C, Forster A C, Bestor T H, Jaenisch R (1993) DNA Methylation, Genomic Imprinting, and Mammalian Development. Cold Spring Harb. Symp. Quant. Biol. 58, 297-305.

Li E, Beard C, Jaenisch R. (1993) Role for DNA methylation in genomic imprinting. Nature 366, 362-365.

Li E, Bestor T H, Jaenisch R (1992) Targeted Mutation of the DNA Methyltransferase Gene Results in Embryonic Lethality. Cell 69, 915-926.

Lin I G, Tomzynski T J, Ou Q, Hsieh C L (2000) Modulation of DNA binding protein affinity directly affects target site demethylation. Mol. Cell. Biol. 20, 2343-2349.

Lisitsyn N, Lisitsyn N, Wigler M. (1993) Cloning the Differences Between Two Complex Genomes. Science 259, 946-951.

Litt M D, Hornstra I K, Yang T P 1996) In Vivo Footprinting and High-Resolution Methylation Analysis of the Mouse Hypoxanthine Phosphoribosyltransferase Gene 5′ Region on the Active and Inactive X Chromosomes. Mol. Cell. Biol. 16, 6190-9699.

Magewu A N, Jones P A (1994) Ubiquitous and Tenacious Methylation of the CpG Site in Codon 248 of the p53 Gene May Explain Its Frequent Appearance as a Mutational Hot Spot in Human Cancer. Mol. Cell. Biol. 14, 4225-4232.

Mertineit C, Yoder J A, Taketo T, Laird D W, Trasler J M, and Bestor T H (1998) Sex-specific exons control DNA methyltransferase in mammalian germ cells. Development 125, 889-897.

Petronis A, Gottesman I I, Crow T J, DeLisi L E, Klar A J, Macciardi F, McInnis M G, McMahon F J, Paterson A D, Skuse D, Sutherland G R (2000) Psychiatric epigenetics: a new focus for the new century. Mol. Psychiatry 5, 342-346.

Piras G, El Kharroubi A, Kozlov S, Escalante-Alacalde D, Hernandez L, Copeland N G, Gilbert D J, Jenkins N A, Stewart C L (2000) Zac1 (Lot1 ), a Potential Tumor Suppressor Gene, and the Gene for epsilon-Sarcoglycan Are Maternally Imprinted Genes: Identification by a Substractive Screen of Novel Uniparental Fibroblast Lines. Mol. Cell. Biol. 20, 3308-3315.

Panning B, Jaenisch R (1996) DNA hypomethylation can activate Xist expression and silence X-linked genes. Genes Dev. 10, 1991-2002.

Schübeler D, Lorincz M C, Cimbora D M, Telling A, Feng Y Q, Bouhassira E E, Groudine M (2000) Genomic Targeting of Methylated DNA: Influence of Methylation on Transcription, Replication, Chromatin Structure, and Histone Acetylation. Mol. Cell. Biol. 20, 9103-9112.

Shibata H, et al. (1994) Genetic mapping and systematic screening of mouse endogenously imprinted loci detected with restriction landmark genome scanning method (RLGS). Mamm. Genome 5, 797-800.

Smit A F A (1999) Interspersed repeats and other mementos of transposable elements in mammalian genomes. Curr. Opin. Genet. Dev. 9, 657-663.

Stewart, F J, Raleigh, E A (1998) Dependence of McrBC Cleavage on Distance Between Recognition Elements. Biol. Chem. 379, 611-616.

Turker M S, Swisshelm K, Smith A C, Martin G M (1989) A Partial Methylation Profile for a CpG Site is Stably Maintained in Mammalian Tissues and Cultured Cell Lines. J. Biol. Chem. 264, 11632-11636.

Ushijima T, Morimura K, Hosoya Y, Okonogi H, Tatematsu M, Sugimura T, Nagao M (1997) Establishment of methylation-sensitive-representational difference analysis and isolation of hypo- and hypermethylated genomic fragments in mouse liver tumors. Proc. Natl. Acad. Sci. USA 94, 2284-2289.

Venter J C, et al. (2001) The Sequence of the Human Genome. Science 291, 1304-1350.

Warnecke P M, Bestor T H (2000) Cytosine methylation and human cancer. Curr. Op. Oncology 12, 68-73.

Walsh C P, Bestor T H (1999) Cytosine methylation and mammalian development. Genes & Devel. 13, 26-34.

Walsh C P, Chaillet J R, Bestor T H (1998) Transcription of IAP endogenous retroviruses is constrained by cytosine methylation. Nature Genetics 20, 116-117.

Xu G -L, Bestor T H, Bourc'his D, Hsieh C -L, Tommerup N, Bugge M, Hulten M, Qu X, Russo J J, Viegas-Péquignot E (1999) Chromosome instability and immunodeficiency syndrome caused by mutations in a DNA methyltransferase gene. Nature 402, 187-191.

Yoder J A, Bestor T H (1998) A candidate mammalian DNA methyltransferase related to pmt1p of fission yeast. Hum. Mol. Gen. 7, 279-284.

Yoder J A, Walsh C P, Bestor T H (1997) Cytosine methylation and the ecology of intragenomic parasites. Trends Genet. 13, 335-340.

Zhu X, Deng C, Kuick R, Yung R, Lamb B, Neel J V, Richardson B, Hanash S (1999) Analysis of human peripheral blood T cells and single-cell-derived T cell clones uncovers extensive clonal CpG island methylation heterogeneity throughout the genome. Proc. Natl. Acad. Sci. 96, 8058-8063.

What is claimed is:

1. A method for detecting the presence of differential methylation between DNA comprising all or a portion of a promoter from a first source and the corresponding DNA from a second source, which method comprises the steps of:
(a) (i) contacting an agent that degrades methylated DNA with a sample of the DNA from the first source, under suitable conditions, so as to degrade methylated DNA in the first sample, and (ii) contacting an agent that degrades unmethylated DNA with a sample of the DNA from the second source, under suitable conditions, so as to degrade unmethylated DNA in the second sample;
(b) contacting the resulting samples with each other under conditions permitting reannealing between the DNA strands therein, so as to permit the formation of a hybrid DNA duplex comprising a DNA strand from the first source and a DNA strand from the second source, should both such strands be present; and
(c) detecting the formation of any such hybrid DNA duplex, such formation indicating the presence of differential methylation between the DNA from the first source and the corresponding DNA from the second source.

2. The method of claim 1, further comprising the step of modifying the DNA of parts (i) and (ii) resulting from step (a) with a first and second moiety, respectively, so as to prevent, in step (b), the formation of a DNA duplex consisting of DNA strands from the first source or of a DNA duplex consisting of DNA strands from the second source.

3. The method of claim 2, wherein the modification of at least one sample resulting from step (a) comprises modifying the DNA in at least one sample with a moiety which facilitates the isolation of hybrid DNA duplexes formed in step (b).

4. The method of claim 3, wherein the moiety is biotin.

5. The method of claim 1, further comprising the step of determining the nucleic acid sequence of a hybrid DNA duplex whose presence is detected in step (c).

6. The method of claim 5, further comprising the step of identifying the methylated nucleotide residues of one or both strands of the hybrid DNA duplex whose sequence is determined.

7. The method of claim 1, wherein the first and second sources of DNA are a cell from a first tissue of a subject and a cell from a second tissue of that subject, respectively.

8. The method of claim 1, wherein the first and second sources of DNA are a cell from a normal tissue and a cell from that tissue in a diseased state, respectively.

9. The method of claim 1, wherein the first and second sources of DNA are both chromosomes of a chromosome pair.

10. The method of claim 1, wherein each of the DNA samples from the first and second sources consists of all or a portion of a promoter.

11. The method of claim 10, wherein the promoter is a promoter for a tumor suppressor gene.

12. The method of claim 10, wherein the promoter is a promoter for an oncogene.

13. The method of claim 1, wherein the agent that degrades methylated DNA is McrBC.

14. The method of claim 1, wherein the agent that degrades unmethylated DNA comprises a methylation-sensitive restriction endonuclease.

15. The method of claim 14, wherein the methylation-sensitive restriction endonuclease is selected from the group consisting of HpaII, HhaI, MaeII, BstUI and AciI.

16. The method of claim 1, wherein the agent that degrades unmethylated DNA comprises a plurality of methylation-sensitive restriction endonucleases.

17. The method of claim 15, wherein the plurality of methylation-sensitive restriction endonucleases is selected from the group consisting of HpaII, HhaI, MaeII, BstUI and AciI.

18. The method of claim 1, wherein the DNA from the first and second sources is human DNA.

19. A method for determining whether a promoter is methylated, which method comprises the steps of:
(a) (i) contacting an agent that degrades unmethylated DNA with a first sample of DNA comprising all or a portion of the promoter, under suitable conditions, so as to degrade unmethylated DNA in the sample, and (ii) contacting an agent that degrades methylated DNA with a second sample of DNA corresponding to the first, under suitable conditions, so as to degrade methylated DNA in the sample, wherein the DNA in the second sample is known to be unmethylated;
(b) contacting the resulting samples with each other under conditions permitting reannealing between the DNA strands therein, so as to permit the formation of a hybrid DNA duplex comprising a DNA strand from the first sample and a DNA strand from the second sample, should both such strands be present; and
(c) detecting the formation of any such hybrid DNA duplex, such formation indicating the presence of differential methylation between the DNA from the first sample and the corresponding DNA from the second, and hence indicating that the promoter is methylated.

20. The method of claim 19, further comprising the step of modifying the DNA of parts (i) and (ii) resulting from step (a) with a first and second moiety, respectively, so as to prevent, in step (b), the formation of a DNA duplex consisting of DNA strands from the first sample or of a DNA duplex consisting of DNA strands from the second sample.

21. The method of claim 20, wherein the modification of at least one sample resulting from step (a) comprises modifying the DNA in at least one sample with a moiety which facilitates the isolation of hybrid DNA duplexes formed in step (b).

22. The method of claim 21, wherein the moiety is biotin.

23. The method of claim 19, further comprising the step of determining the nucleic acid sequence of a hybrid DNA duplex whose presence is detected in step (c).

24. The method of claim 23, further comprising the step of identifying the methylated nucleotide residues of one or both strands of the hybrid DNA duplex whose sequence is determined.

25. The method of claim 19, wherein the agent that degrades methylated DNA is McrBC.

26. The method of claim 19, wherein the agent that degrades unmethylated DNA comprises a methylation-sensitive restriction endonuclease.

27. The method of claim 26, wherein the methylation-sensitive restriction endonuclease is selected from the group consisting of HpaII, HhaI, MaeII, BstUI and AciI.

28. The method of claim 19, wherein the agent that degrades unmethylated DNA comprises a plurality of methylation-sensitive restriction endonucleases.

29. The method of claim 28, wherein the plurality of methylation-sensitive restriction endonucleases is selected from the group consisting of HpaII, HhaI, MaeII, BstUI and AciI.

30. The method of claim 19, wherein the first and second samples are from a human being.

31. The method of claim 19, wherein the first sample is from a human being known to be afflicted with a disorder.

32. The method of claim 31, wherein the disorder is cancer.

33. The method of claim 19, wherein the promoter is a promoter for a tumor suppressor gene.

34. The method of claim 19, wherein the promoter is a promoter for an oncogene.

35. The method of claim 19, wherein the first and second samples consist of all or a portion of a promoter.

36. A method for determining whether a promoter is methylated, which method comprises the steps of:
(a) (i) contacting a first sample of DNA comprising all or a portion of the promoter with an agent that degrades unmethylated DNA, under suitable conditions, so as to degrade unmethylated DNA in the sample;
(b) contacting the resulting first sample with a second sample of corresponding DNA, known to be unmethylated, under conditions permitting reannealing between the DNA strands therein, so as to permit the formation of a hybrid DNA duplex comprising a DNA strand from the first sample and a DNA strand from the second sample, should both such strands be present; and
(c) detecting the formation of any such hybrid DNA duplex, such formation indicating the presence of differential methylation between the DNA from the first sample and the corresponding DNA from the second, and hence indicating that the promoter is methylated.

37. The method of claim 36, further comprising the step of modifying the DNA of the first and second samples with a first and second moiety, respectively, so as to prevent, in step (b), the formation of a DNA duplex consisting of DNA strands from the first sample or of a DNA duplex consisting of DNA strands from the second sample.

38. The method of claim 37, wherein the modification comprises modifying the DNA in at least one sample with a moiety which facilitates the isolation of hybrid DNA duplexes formed in step (b).

39. The method of claim 38, wherein the moiety is biotin.

40. The method of claim 36, further comprising the step of determining the nucleic acid sequence of a hybrid DNA duplex whose presence is detected in step (c).

41. The method of claim 40, further comprising the step of identifying the methylated nucleotide residues of a strand of the hybrid DNA duplex whose sequence is determined.

42. The method of claim 36, wherein the agent that degrades unmethylated DNA comprises a methylation-sensitive restriction endonuclease.

43. The method of claim 42, wherein the methylation-sensitive restriction endonuclease is selected from the group consisting of HpaII, HhaI, MaeII, BstUI and AciI.

44. The method of claim 36, wherein the agent that degrades unmethylated DNA comprises a plurality of methylation-sensitive restriction endonucleases.

45. The method of claim 44, wherein the plurality of methylation-sensitive restriction endonucleases is selected from the group consisting of HpaII, HhaI, MaeII, BstUI and AciI.

46. The method of claim 36, wherein the first and second samples are from a human being.

47. The method of claim 36, wherein the first sample is from a human being known to be afflicted with a disorder.

48. The method of claim 47, wherein the disorder is cancer.

49. The method of claim 36, wherein the promoter is a promoter for a tumor suppressor gene.

50. The method of claim 36, wherein the promoter is a promoter for an oncogene.

51. The method of claim 36, wherein the first and second samples consist of all or a portion of a promoter.
